(19) United States
(12) Patent Application Publication
(10) Pub. No.: US 2012/0157695 A1
(43) Pub. Date: Jun. 21, 2012

(54) Process for the Distillative Purification of Fluoroethylene Carbonate

(75) Inventors: Alain Lambert, Beauvechain (BE); Christoph Sommer, Neckarsulm (DE); Harald Krueger, Bad Wimpfen (DE); Jens Olschimke, Hannover (DE)

(73) Assignee: SOLVAY FLUOR GMBH, Hannover (DE)

(21) Appl. No.: 13/390,541

(22) PCT Filed: Aug. 17, 2010
(86) PCT No.: PCT/EP2010/061973

§ 371 Date: Feb. 15, 2012

(30) Foreign Application Priority Data
Aug. 20, 2009 (EP) 09168329.2

(51) Int. Cl. C07D 317/36; B01D 3/10

(52) U.S. Cl. 549/229; 203/81; 203/77; 203/74; 203/82; 203/75; 203/41

(57) ABSTRACT

Crude fluoroethylene carbonate obtained by the fluorination of ethylene carbonate and elemental fluorine containing not more than 5% by weight of HF is purified by at least two subsequent distillation steps. The bulk of HF can be removed, if desired, in a preliminary HF removal step, e.g., by stripping, before performing the distillation. Further, if desired, a second HF removal step can be performed by contacting the crude mixture or the distillate obtained after the first distillation step with an adsorbent for HF, e.g., silica gel. The distillation can be performed batch wise. It is preferred to perform the distillation continuously. It yields purified fluoroethylene carbonate with an HF content of equal to or less than 30 ppm. The purified fluoroethylene carbonate can be applied as solvent additive for lithium ion batteries.

This application claims priority to European application No. 09168329.2 filed Aug. 20, 2009, the whole content of this application being incorporated herein by reference for all purposes.

The present application concerns a process for purifying fluoroethylene carbonate by distillation.

Fluoroethylene carbonate (“F1EC”), also known as monofluoroethylene carbonate or 4-fluoro-1,3-dioxolane-2-one, is suitable as solvent or solvent additive for lithium ion batteries. It can be prepared from the respective unsubstituted ethylene carbonate by electro fluorination as described by H. Ishii et al. in J. Chem. Soc., Chem. Comm. (2000), pages 1617 and 1618. A preferred method provides for the reaction with elemental fluorine. This is described for example in JP-A 2000-309583 where the reaction is performed with a melt of 1,3-dioxolane-2-one (ethylene carbonate; “EC”) or its solution in anhydrous fluoride. Optionally, an inert solvent like perfluorohexane can be present; in this case, a suspension of 1,3-dixolane-2-one is formed. The desired product is isolated by a first distillation to remove HF, by a treatment with alkaline water, drying, another distillation (product with a purity of 90% or more is obtained hereby) and several recrystallizations. According to US patent application 2006-0036102, ethylene carbonate is dissolved in F1EC and then contacted with fluorine. To the reaction mixture, acetone and potassium carbonate were added, solids were then removed, and thereafter, the resulting solution was distilled several times under vacuum. According to U.S. Pat. No. 7,268,238, the raw product of the reaction between ethylene carbonate and fluorine was first treated two times in a distillation column for HF removal, then distilled twice to further purify it.

Subject of the present invention is to provide a simple, energy-saving process which yields highly pure fluoroethylene carbonate without the need for performing recrystallization.

According to the present invention, a mixture comprising fluoroethylene carbonate, ethylene carbonate, higher fluorinated ethylene carbonate or carbonates and hydrogen fluoride and optionally trace impurities (for example, trifluoroethylene carbonate) is distilled in at least two distillation steps wherein the reaction mixture which is fed to the first distillation step contains not more than 5% by weight of HF. Preferably, the reaction mixture which is fed to the first distillation column contains nor more than 1% by weight of HF.

The wording “at least two distillation steps” denotes passing the mixture at least twice through a distillation column. According to one embodiment, this is one distillation column through which the mixture to be separated is passed at least twice. This embodiment can be performed in a batch wise distillation.

According to another embodiment, the at least two distillation steps are performed in at least two distillation columns. This embodiment is especially suitable for performing a continuous distillation process.

If the raw reaction mixture is obtained by electro fluorination, it is advisable to remove any solids by a respective treatment, e.g. by filtration.

The purified fluoroethylene carbonate obtained by the process of the present invention is so pure, especially in view of the HF content, that no recrystallization is needed. In this manner, starting with a raw product containing ethylene carbonate, 4,5-cis and 4,5-trans difluoroethylene carbonate and 4,4-difluoroethylene carbonate and HF, a purified fluoroethylene carbonate can be obtained which, if at all, contains only traces of trans-4,5- difluoroethylene carbonate and 4,4-difluoroethylene carbonate, and which comprises, if at all, only traces of cis-4,5-difluoroethylene carbonate. Typically, the content of each of trans-4,5- difluoroethylene carbonate, cis-4,5-difluoroethylene carbonate and 4,4-difluoroethylene carbonate is less than 20 ppm.

The initial content of HF in the raw product leaving the fluorination reactor can vary. The substitution of a fluorine atom for a hydrogen atom is accompanied by the formation of one molecule of HF per exchanged hydrogen atom. Besides, it is known that hydrogen fluoride can be used as solvent in such reactions. Thus, the reaction mixture leaving the reactor may comprise up to 10 or even up to 20% by weight and, if HF was used as solvent, even much more

HF.

If the reaction mixture leaving the fluorination reactor or fluorination reactors contains more than 5% by weight of HF, the content of HF is reduced in a preliminary HF removal step to an amount in the reaction mixture of not more than 5% by weight. HF can be removed, for example, by washing the raw product with water or by removing HF by stripping the raw product, for example with an inert gas, especially nitrogen or carbon dioxide. The preliminary HF removal step is not a distillation. According to one embodiment, if the content of HF in the reaction mixture leaving the fluorination reactor or fluorination reactors is equal to lower than 5% by weight in the reaction mixture, the HF content is not reduced in a preliminary HF removal step. In this embodiment, the raw material is directly distilled in two steps.

According to another embodiment, at least a part of the HF in the raw product mixture is removed in a preliminary HF removal step before distillation is performed such that the content is equal to lower than 5% by weight. Preferably, in the preliminary HF removal step, the content of HF is reduced to equal to or less than 2% by weight, more preferably to equal to or less than 1% by weight, and still more preferably, to equal to or less than 0.5% by weight of the reaction mixture.

It is possible to remove the bulk of HF in a first preliminary HF removal step, especially as described above, and then to remove residual HF in a second HF removal step. This second HF removal step is preferably performed using a solid adsorbent or a liquid absorbent. A preferred absorbent comprises SiO₂; silica gel (for example, in bead form) is especially preferred. If desired, a filter containing silica gel particles can be applied through which the raw material can be passed continuously. This adsorbent reacts with HF under formation of water and SiF₄. Water was found to cause side reactions with certain fluorinated organic carbonates. Thus, the initial removal of bulk HF by stripping, the subsequent removal of HF using silica and the additional distillation provide a perfect combination because stripping can be performed without any water being formed, the amount of water formed during the additional treatment with silica is so small that no side reactions take place, and the additional distillation, together with the preceding HF removal steps, provide a highly pure product while the yield of the desired product is exceedingly high.

In one embodiment, the process comprises a step wherein the second HF removal step is performed before the two distillation steps. In another embodiment, the process comprises a step wherein the second HF removal step is performed between the distillation steps.

Neither the first preliminary HF removal step nor the second HF removal step are performed by distillation, but, as described above, by stripping, adsorption, washing with water or alkaline aqueous solutions or other means.

The process according to the present invention can be performed batch wise or continuously.

If a batch distillation is performed, the pressure in the distillation steps is preferably equal to or lower than 100 mbar (abs). It is preferably equal to or lower than 15 mbar (abs.), especially preferably, equal to or lower than 5 mbar (abs.). Often, in double batch distillations, the first batch distillation is advantageously performed at a higher pressure than the second one. Preferably, the pressure at the top of the column in the first batch distillation is equal to or lower than 10 mbar (abs), and in the second batch distillation, it is performed at a lower pressure than at the first distillation of equal to lower than 5 mbar (abs.). The pressure is preferably equal to or greater than 0.5 mbar (absolute).

A process is especially preferred wherein a reaction mixture comprising fluoroethylene carbonate, ethylene carbonate, higher fluorinated carbonates and HF with an HF content with more than 5% by weight of HF is subjected to a stripping process to reduce the content of HF to obtain a reaction mixture containing fluoroethylene carbonate, ethylene carbonate, higher fluorinated carbonates and HF with an HF content with not more than 5% by weight of HF, and the resulting reaction mixture is distilled in at least two distillation steps. The preferred embodiments of this process are those which are explained in detail above and below. It is, for example, preferred to perform the distillation steps continuously. It is also preferred to perform the distillation in at least two columns, and more preferably, to perform it in two columns.

The invention will now be described in detail for the preferred embodiment which provides for a continuous process.

The distillation is performed in at least two steps. As mentioned above, the continuous distillation is preferably performed in at least two consecutive distillation columns.

In the first distillation step, a mixture of substances with a lower boiling point (for example, HF and difluorinated ethylene carbonates) is drawn off from the top; the higher boiling constituents (mostly ethylene carbonate and monofluoroethylene carbonate) are drawn off from the bottom and are fed into the second distillation step. Often, the pressure at the top of the column of the first distillation step is equal to or lower than 100 mbar (abs). Preferably, the pressure at the top of the column of the first distillation step is equal to or lower than 75 mbar (abs.). Preferably, it is equal to or higher than 10 mbar (abs.). A pressure at the top of the column of the first distillation step in the range between 10 and 50 mbar (abs.) is especially preferred.

The mixture of substances with a lower boiling point drawn off from the top of the column of the first distillation step, mainly HF and difluoroethylene carbonates, can be separated if desired. For example, HF can be removed by washing the mixture with water or, which is highly preferred, by stripping the mixture with an inert gas. The remaining difluoroethylene carbonates can be separated by distillation. Alternatively, the mixture from the top of the column of the first distillation step can be separated into the different compounds simply by distillation without any other treatment like washing or stripping. Difluorinated ethylene carbonates are valuable side products because they can be applied as additive for lithium ion battery solvents. If desired, they can be dumped or burned. Any recovered hydrogen fluoride also is a valuable product per se.

In the second column, the bottom product of the first column is distilled. Preferably, the pressure at the top of the column of the second distillation step is equal to or lower than 50 mbar (abs.). More preferably, the pressure at the top of the second column is equal to or lower than 30 mbar (abs.). Preferably, the pressure at the top of the column of the second distillation step is equal to or higher than 5 mbar (abs). The conditions in the column are selected so that in the bottom a mixture of ethylene carbonate and monofluoroethylene carbonate is formed; thereby, the degree of purity of monofluoroethylene carbonate drawn off from the top is increased.

At the top of the column of the second distillation step, highly pure fluoroethylene carbonate is obtained. The purity of the top product is so high that it can be applied immediately for any desired purpose, notably as solvent or solvent additive for lithium ion batteries. The content of HF in the purified fluoroethylene carbonate is equal to or lower than 30 ppm by weight, preferably equal to or lower than 20 ppm by weight. The examples demonstrate that an even lower HF content can be achieved, e.g. equal to or lower than 10 ppm. The content of cis-difluoroethylene carbonate is below 20 ppm. Usually, the amount of each of trans-difluoroethylene carbonate and 4,4-difluoroethylene carbonate is below 20 ppm.

Often, the first distillation step is performed in a column having 10 to 50 theoretical stages. Often, the second distillation step is performed in a column having 10 to 30 theoretical stages. If after purification, F1EC is obtained which does not have a desired degree of purity, e.g. the HF content is greater than 30 ppm, one or both distillations can be performed in a column or columns with a greater number of theoretical plates such that the desired purity, preferably equal to or less than 30 ppm are achieved.

If desired, a third distillation step can be performed to further purify the fluoroethylene carbonate obtained in the second distillation step. The preferred ranges of the pressure in the third distillation step and any further distillation step correspond to the preferred ranges of the pressure in the second distillation step.

Distillation residues contain F1EC and EC and can be returned to the reaction vessel in which the fluorination reaction between EC and fluorine is performed, or they can be added to the raw material before the first distillation.

As mentioned above, the raw reaction mixture (obtained by the reaction of starting from ethylene carbonate and fluorine, optionally in the presence of HF, fluoroethylene carbonate or both as solvent) can be treated by a stripping process to reduce the HF content to 2% by weight or lower. A second treatment to remove HF comprises contacting the mixture with silica gel. This second HF removal step can be performed before the distillation steps, or it can be performed after the first distillation and before the second distillation step. If a treatment with an absorbent is performed, it is preferred to perform it before the first distillation step.

The mixture of monofluoroethylene carbonate and ethylene carbonate from the bottom of the second column can be recycled to the fluorination reactor. It was already mentioned above that, according to US patent application 2006-0036102, fluoroethylene carbonate can be applied as solvent for ethylene carbonate.

The columns which are applied in the distillation steps are known in the art. Usually, in vacuum distillation, columns with bulk or structured packing are applied.

The process according to the invention provides purified fluoroethylene carbonate without the need for purification by additional recrystallization steps or by extensive distillation steps. Aqueous workup which is accompanied by loss of material is avoided.

Should the disclosure of any patents, patent applications, and publications which are incorporated herein by reference conflict with the description of the present application to the extent that it may render a term unclear, the present description shall take precedence.

The following examples are intended to describe the invention further without limiting it.

### EXAMPLE 1

### Purification of Fluoroethylene Carbonate by Continuous Distillation

Abbreviations:

EC: Ethylene carbonate

F1EC: Monofluoroethylene carbonate

CIS-F2EC: cis-4,5-Difluoroethylene carbonate

4,4-F2EC: 4,4-Difluoroethylene carbonate

TR-F2EC: trans-4,4-Difluoroethylene carbonate

E −n means 10⁻ⁿ (for example: E⁻⁴ is 10⁻⁴)

Apparatus: The apparatus includes two columns K1 and K2. K1 has 20 to 30 theoretical stages with feed (delivered in a feed line F-K1) entering the column in a stage above the lower third. K2 has 12 to 20 theoretical stages; the feed which is the bottom product of K1 is delivered via a line B1 and enters the column K2 in a stage above the middle of the column.

The distillate from the top of K1 is drawn off in a line D1. A part of the distillate is returned to K1 via a line REF-1.

The distillate from the top of K2 is drawn off in a line D2; a part of the distillate is returned to K2 via a line REF-2. The bottom product of K2 is drawn off in line B2.

Feed: The feed is the crude reaction mixture from the reaction between ethylene carbonate and a F₂/N₂ mixture up to a conversion of 50 mol % of the ethylene carbonate from which the bulk of HF contained is removed by stripping; further HF is removed by contact with silica gel. The HF content in the feed is below 300 ppm and is neglected in the following. The temperature of the feed in feed line F-K1 is 106.8° C., the total mass flow rate is 77.8 kg/hr.

The temperature at the top of column K1 is slightly above 40° C., the pressure is about 25 mbar (abs). The temperature at the top of column K2 is about 80° C., the pressure is about 8 mbar (abs). The temperature in the bottom of both columns is slightly above 130° C.

In table 1, the mass fraction (in % by weight) of the compounds contained in the fluid passing through lines F-K1 (feed line) and D2 (the line through which the final F1EC is drawn off) are compiled:

TABLE 1
Composition of feed stream and distillate

| Stream in Line | F-K1 | D2 |
|---|---|---|
| EC | 0.463 | 5.13E−6 |
| F1EC | 0.424 | 0.999967 |
| CIS-F2EC | 0.028 | 2.79E−5 |
| 4,4-F2EC | 0.019 | 0.0 |
| TR-F2EC | 0.066 | 0.0 |

Table 1 demonstrates that through the line D2, a highly purified fluoroethylene carbonate is withdrawn. Impurities are in the lower ppm range.

### EXAMPLE 2

### Batch Distillation of Monofluoroethylene Carbonate

Apparatus used:

The distillation was performed using a steam-heated boiler with mechanical stirrer and a column of 4 sections connected to the vessel. The column is 4 m long, filled with glass random packaging and connected with a condenser located directly on top of the column.

The starting material (850 l) was obtained from the reaction of ethylene carbonate (dissolved in F1EC) and elemental fluorine, diluted in nitrogen. Most of the HF formed was removed by stripping. The composition of the starting material before distillation was (figures given in weight -%):

EC: 34%

F1EC: 58%

F2EC: 8% (TR-F2EC 3%, 4,4-F2EC 1%, CIS-F2EC 4%).

HF: <0.2%

Before distillation starts, 10 kg of silica gel were added to the starting material in the boiler to neutralize HF. Before starting the distillation, a degassing is performed by lowering the pressure to about 1 mbar (abs). Hereby, dissolved gases and water formed (from a reaction between the silica gel and HF) are removed from the starting material. Condensable constituents of the gas stream obtained in this step were removed to protect the vacuum pump.

The starting material in the boiler was heated to about 125° C. The pressure at the top of the column was 3.5 mbar (abs), the temperature at the top was about 73° C.

At the beginning of the distillation, the F2EC isomers reach the top of the column with high concentration. They can be disposed.

The distillate was collected in a separate storage tank when the content of the F2EC isomers was below 2% by weight. The collection of the distillate was terminated as soon as the content of EC in the distillate reached 2% by weight. The composition of the liquid in the storage tank was slightly less than 2% by weight of the F2EC isomers, 97.5% by weight of F1EC and 0.5% by weight of EC. The liquid remaining in the boiler had a composition of about 10% by weight of F1EC and about 90% by weight of EC, was removed from the boiler and added to the starting material of another batch to produce F1EC from EC and fluorine. The silica gel was dumped.

The storage tank contained about 500 liters of distillate. It was returned to the boiler and 5 kg fresh silica gel was added. This time, no degassing was performed. The liquid in the boiler was heated to 125° C., and the pressure at the top of the column was 1.5 mbar (abs). The distillate recovered at the beginning contained much CIS-F2EC and was returned to the raw material from another fluorination for redistillation.

The distillate was collected in a fine product storage tank as soon as it contained >99.1% by weight of F1EC. The remaining liquid (which was later added to the starting material of another fluorination reaction of EC) in the boiler at the end of the distillation contained about 80% by weight of F1EC and about 20% by weight of EC.

The total yield of isolated fine product was about 36% by weight after the two distillation steps.

1. A process for preparing purified fluoroethylene carbonate, comprising distilling a reaction mixture comprising fluoroethylene carbonate, ethylene carbonate, higher fluorinated carbonates and HF in at least two distillation steps, wherein the reaction mixture which is fed to a first distillation step contains not more than 5% by weight of HF.

2. The process of claim 1 wherein the reaction mixture which is fed to the first distillation step contains not more than 1% by weight of HF.

3. The process according to claim 1 wherein the distillation in the at least two distillation steps is performed continuously.

4. The process according to claim 3 wherein the reaction mixture is fed continuously into a first column for carrying out the first distillation step wherein lower boiling compounds including HF, difluorinated organic carbonates and higher fluorinated organic carbonates are drawn off from the top of said first column, and wherein higher boiling compounds including ethylene carbonate and fluoroethylene carbonate are drawn off from the bottom of the first column, and wherein said drawn-off higher boiling compounds are fed to a second column for carrying out a second distillation step wherein pure monofluoroethylene carbonate is drawn off from the top of the second column.

5. The process according to claim 3 wherein the distillation in the first distillation step is performed in a column at a pressure at the top of the column of equal to or lower than 100 mbar (abs).

6. The process according to claim 3 wherein the distillation in a second distillation step is performed in a column at a pressure at the top of the column of equal to or less than 50 mbar (abs).

7. The process according to claim 1 wherein the purified fluoroethylene carbonate comprises equal to or less than 30 ppm HF.

8. The process according to claim 1 wherein the distillation in the at least two distillation steps is performed batch wise in one distillation column.

9. The process according to claim 8 wherein the pressure at the top of the column for carrying out the first distillation step is higher than the pressure at the top of the column for carrying out a second distillation step, and wherein the pressure at the top of the column for carrying out the first distillation step is equal to or lower than 10 mbar (abs.), and wherein the pressure at the top of the column for carrying out the second distillation step equal to or lower than 5 mbar (abs).

10. The process of claim 1 wherein the distillation in the at least two distillation steps is performed in at least two distillation columns.

11. The process of claim 10 wherein the distillation in the at least two distillation steps is performed in two distillation columns.

12. The process of claim 1, further comprising:
subjecting a crude reaction mixture comprising fluoroethylene carbonate, ethylene carbonate, higher fluorinated carbonates and HF with an HF content with more than 5% by weight of HF to a stripping process to reduce the content of HF and to obtain the reaction mixture containing not more than 5% by weight of HF and which is distilled in the at least two distillation steps.

13. The process of claim 12, wherein said stripping is carried out with an inert gas selected from the group consisting of nitrogen and carbon dioxide.

14. The process of claim 12, further comprising a second HF removal step using a solid adsorbent or a liquid absorbent, said second HF removal step being performed before the at least two distillation steps or between the distillation steps.

15. The process of claim 14, wherein said second HF removal step uses a solid adsorbent comprising SiO₂.

16. The process of claim 1, further comprising an HF removal step which is performed by contacting a crude reaction mixture obtained by fluorination of ethylene carbonate and elemental fluorine or contacting a distillate obtained after the first distillation step with an adsorbent for HF.

17. The process of claim 16, wherein said second HF removal step uses an adsorbent comprising silica gel.
